(19) United States
(12) Patent Application Publication
(10) Pub. No.: US 2005/0096290 A1
(43) Pub. Date: May 5, 2005

(54) 5'-and 3'-capped aptamers and uses therefor

(76) Inventors: Anthony P. Adamis, Boston, MA (US); David Shima, Boston, MA (US); Francine Wincott, Baltimore, MD (US); Perry Calias, Melrose, MA (US)

Correspondence Address: EYETECH PHARMACEUTICALS, INC., 3 TIMES SQUARE 12TH FLOOR, NEW YORK, NY, 10036, US

(21) Appl. No.: 10/913,259

(22) Filed: Aug. 6, 2004

Related U.S. Application Data
(60) Provisional application No. 60/493,500, filed on Aug. 8, 2003.

(51) Int. Cl. A61K 48/00; C12Q 1/68; C07H 21/02

(52) U.S. Cl. 514/44; 435/6; 536/23.1

(57) ABSTRACT

The invention provides compositions and methods for the treating disease using aptamers having 5′-5′ and 3′-3′ inverted nucleotide capped ends. In particular, the invention provides 5′-5′ and 3′-3′ capped anti-VEGF aptamers for the treatment of neovascularization-related diseases and disorders including age-related macular degeneration.

### RELATED APPLICATIONS

This application claims priority to U.S. Provisional Application Ser. No. 60/493,500, filed Aug. 8, 2003, which is hereby incorporated in its entirety by reference.

### FIELD OF THE INVENTION

The invention relates to angiogenesis and neovascularization. More specifically, the invention relates to anti-vascular endothelial growth factor (anti-VEGF) aptamers that inhibit neovascularization or angiogenesis, and the treatment of diseases associated with neovascularization or angiogenesis.

### BACKGROUND OF THE INVENTION

Angiogenesis, or neovascularization, is the process by which new blood vessels develop from existing endothelium. Normal angiogenesis plays an important role in a variety of processes including embryonic development, wound healing and several components of female reproductive function, however angiogenesis is also associated with certain pathological conditions. Undesirable or pathological angiogenesis has been associated with certain disease states including proliferative retinopathies, rheumatoid arthritis, psoriasis and cancer (see Fan et al. (1995) Trends Pharmacol. Sci. 16: 57; and Folkman (1995) Nature Medicine 1: 27). Indeed the quantity of blood vessels in tumor tissue is a strong negative prognostic indicator in breast cancer (Weidner et al. (1992) J. Natl. Cancer Inst. 84:1875-1887), prostate cancer (Weidner et al. (1993) Am. J. Pathol. 143:401-409), brain tumors (Li et al. (1994) Lancet 344:82-86), and melanoma (Foss et al. (1996) Cancer Res. 56:2900-2903). Furthermore, the alteration of vascular permeability is thought to play a role in both normal and pathological physiological processes (Cullinan-Bove et al. (1993) Endocrinol. 133: 829; Senger et al. (1993) Cancer and Metastasis Reviews 12: 303).

Various growth factors that are capable of inducing angiogenesis have been identified to date. These include basic and acidic fibroblast growth factors (aFGF, bFGF), transforming growth factors alpha and beta (TGFα, TGFβ), platelet derived growth factor (PDGF), angiogenin, platelet-derived endothelial cell growth factor (PD-ECGF), interleukin-8 (IL-8), and vascular endothelial growth factor (VEGF). Among these, VEGF appears to play a key role as a positive regulator of physiological and pathological angiogenesis (reviewed in Brown et al. (1996) Control of Angiogenesis (Goldberg and Rosen, eds.) Birkhauser, Basel, and Thomas (1996) J. Biol. Chem. 271:603-606).

VEGF is a secreted disulfide-linked homodimer that selectively stimulates endothelial cells to proliferate, migrate, and produce matrix-degrading enzymes (Conn et al. (1990) Proc. Natl. Acad. Sci. USA 87:1323-1327); Ferrara and Henzel (1989) Biochem. Biophys. Res. Commun. 161: 851-858); Pepper et al. (1991) Biochem. Biophys. Res. Commun. 181:902-906; Unemori et al. (1992) J. Cell. Physiol. 153:557-562), all of which are processes required for the formation of new vessels. In addition to being the only known endothelial cell specific mitogen, VEGF is unique among angiogenic growth factors in its ability to induce a transient increase in blood vessel permeability to macromolecules (hence its original and alternative name, vascular permeability factor, VPF) (see Dvorak et al. (1979) J. Immunol. 122:166-174; Senger et al. (1983) Science 219:983-985; Senger et al. (1986) Cancer Res. 46:5629-5632). Increased vascular permeability and the resulting deposition of plasma proteins in the extravascular space assists the new vessel formation by providing a provisional matrix for the migration of endothelial cells (Dvorak et al. (1995) Am. J. Pathol. 146:1029-1039). Hyperpermeability is indeed a characteristic feature of new vessels, including those associated with tumors. In addition, compensatory angiogenesis induced by tissue hypoxia is now known to be mediated by VEGF (Levy et al. (1996) J. Biol. Chem. 2746-2753); Shweiki et al. (1992) Nature 359:843-845).

VEGF occurs in four forms (VEGF-121, VEGF-165, VEGF-189, VEGF-206) as a result of alternative splicing of the VEGF gene (Houck et al. (1991) Mol. Endocrin. 5:1806-1814; Tischer et al. (1991) J. Biol. Chem. 266:11947-11954). The two smaller forms are diffusible whereas the larger two forms remain predominantly localized to the cell membrane as a consequence of their high affinity for heparin. VEGF-165 also binds to heparin and is the most abundant form. VEGF-121, the only form that does not bind to heparin, appears to have a lower affinity for VEGF receptors (Gitay-Goren et al. (1996) J. Biol. Chem. 271:5519-5523) as well as lower mitogenic potency (Keyt et al. (1996) J. Biol. Chem. 271:7788-7795). The biological effects of VEGF are mediated by two tyrosine kinase receptors (Flt-1 and Flk-1/KDR) whose expression is highly restricted to cells of endothelial origin (de Vries et al. (1992) Science 255:989-991; Millauer et al. (1993) Cell 72:835-846; Terman et al. (1991) Oncogene 6:519-524). While the expression of both functional receptors is required for high affinity binding, the chemotactic and mitogenic signaling in endothelial cells appears to occur primarily through the KDR receptor (Park et al. (1994) J. Biol. Chem. 269:25646-25654; Seetharam et al. (1995) Oncogene 10:135-147; Waltenberger et al. (1994) J. Biol. Chem. 26988-26995). The importance of VEGF and VEGF receptors for the development of blood vessels has recently been demonstrated in mice lacking a single allele for the VEGF gene (Carmeliet et al. (1996) Nature 380:435-439; Ferrara et al. (1996) Nature 380:439-442) or both alleles of the Flt-1 (Fong et al. (1995) Nature 376:66-70) or Flk-1 genes (Shalaby et al. (1995) Nature 376:62-66). In each case, distinct abnormalities in vessel formation were observed resulting in embryonic lethality.

VEGF is produced and secreted in varying amounts by virtually all tumor cells (Brown et al. (1997) Regulation of Angiogenesis (Goldberg and Rosen, Eds.) Birkhauser, Basel, pp. 233-269). Direct evidence that VEGF and its receptors contribute to tumor growth was recently obtained by a demonstration that the growth of human tumor xenografts in nude mice could be inhibited by neutralizing antibodies to VEGF (Kim et al. (1993) Nature 362:841-844), by the expression of dominant-negative VEGF receptor flk-1 (Millauer et al. (1996) Cancer Res. 56:1615-1620; Millauer et al. (1994) Nature 367:576-579), by low molecular weight inhibitors of Flk-1 tyrosine kinase activity (Strawn et al. (1966) Cancer Res. 56:3540-3545), or by the expression of antisense sequence to VEGF mRNA (Saleh et al. (1996) Cancer Res. 56:393-401). Importantly, the incidence of tumor metastases was also found to be dramatically reduced by VEGF antagonists (Claffey et al. (1996) Cancer Res. 56:172-181).

VEGF inhibitors have broad clinical utility due to the role of VEGF in a wide variety of diseases involving angiogenesis, including psoriasis, ocular disorders, collagen vascular diseases and neoplastic diseases. One type of VEGF inhibitor is nucleic acid-based VEGF ligand termed an aptamer. Aptamers are chemically synthesized short strands of nucleic acid that adopt specific three-dimensional conformations and are selected for their affinity to a particular target through a process of in vitro selection referred to as systematic evolution of ligands by exponential enrichment (SELEX). SELEX is a combinatorial chemistry methodology in which vast numbers of oligonucleotides are screened rapidly for specific sequences that have appropriate binding affinities and specificities toward any target. Using this process, novel aptamer nucleic acid ligands that are specific for a particular target may be created. The SELEX process in general, and VEGF aptamers and formulations in particular, are described in, e.g., U.S. Pat. Nos. 5,270,163, 5,475,096, 5,696,249, 5,670,637, 5,811,533, 5,817,785, 5,958,691, 6,011,020, 6,051,698, 6,147,204, 6,168,778, and 6,426,335, the contents of each of which is specifically incorporated by reference herein. Anti-VEGF aptamers are small stable RNA-like molecules that bind with high affinity to the 165 kDa isoform of human VEGF.

Accordingly, aptamer antagonists of VEGF are useful in the treatment of diseases involving neovascularization. For example, VEGF antagonists have been used to treat neovascular age-related macular degeneration (AMD), a progressive condition characterized by the presence of choroidal neovascularization (CNV) that results in more severe vision loss than any other disease in the elderly population (see Csaky et al. (2003) Ophthalmol. 110: 880-1).

AMD results from damage to the macula, which is the central region of the retina. The eye lens focuses light onto the macula to allow perception of fine details in central vision. Damage to the macula causes central vision deterioration. Risk factors for AMD include heredity, advanced age, blue eyes and white skin. Current treatments for AMD include photodynamic therapy, which combines a systemically administered drug with laser light therapy to the eye. The systemic drug is a photosensitive chemical that is activated to produce singlet-oxygen radicals that close leaky blood vessels. Although this treatment is effective in slowing the progression of the disease as measured by reduction in percent of patients with vision loss, the treatment does not reverse the disease process and few patients have any improvement in their vision.

In contrast, inhibitors of VEGF directly act to block the formation of new blood vessels, reduce the leakiness of vessels, and potentially lead to vessel regression. Accordingly, anti-VEGF aptamers may stop the progression of AMD and help improve vision. Animal models have confirmed that VEGF is capable of inducing choroidal neovascularization, and pharmacologic studies in humans have demonstrated that intravitreally injected anti-VEGF aptamers are effective in treating neovascular age-related macular degeneration (see Fish et al. (2003) Ophthalmol. 110: 979-86). When injected into the vitreous of the eye to treat eye disease involving neovascularization, anti-VEGF aptamers have been conjugated to polyethylene glycol (PEG), which aids in stabilizing the compound (see, e.g., Drolet et al. (2000) Pharm. Res. 17: 1503-10). Accordingly, other anti-VEGF aptamers for the treatment of macular degeneration and other diseases involving neovascularization, are also desirable.

### SUMMARY OF THE INVENTION

It has been observed that aptamers, or nucleic acid ligands, in general, and VEGF aptamers in particular, are most stable, and therefore efficacious when 5′-capped and 3′-capped in a manner which decreases susceptibility to exonucleases and increases overall stability. Accordingly, the invention is based, in part, upon the capping of aptamers in general, and anti-VEGF aptamers in particular, with a 5′-5′ inverted nucleoside cap structure at the 5′ end and a 3′-3′ inverted nucleoside cap structure at the 3′ end.

Thus, in one aspect, the invention provides aptamers, i.e., nucleic acid ligands, that are capped at the 5′ end with a 5′-5-inverted nucleoside cap and at the 3′ end with a 3′-3′ inverted nucleoside cap. In some embodiments, the capped aptamers are RNA aptamers, DNA aptamers, or aptamers having a mixed (i.e., both RNA and DNA) composition.

In another aspect, the invention provides anti-VEGF aptamer compositions. The anti-VEGF aptamers of the invention have both 5′-5′ and 3′-3′ inverted nucleotide cap structures. In some embodiments, the anti-VEGF capped aptamers of the invention are RNA aptamers, DNA aptamers or aptamers having a mixed (i.e., both RNA and DNA) composition.

In one embodiment, the anti-VEGF capped aptamers of the invention include the nucleotide sequence GAAGAAUUGG (SEQ ID NO: 2). In another embodiment, the anti-VEGF capped aptamers of the invention include the nucleotide sequence UUGGACGC (SEQ ID NO: 3). In still another embodiment, the anti-VEGF capped aptamers of the invention include the nucleotide sequence GUGAAUGC (SEQ ID NO: 4). In a particular embodiment, the capped anti-VEGF aptamers of the invention have the sequence:

| | |
|---|---|
| X-5′-5′- | (SEQ ID NO: 1) |
| CGGAAUCAGUGAAUGCUUAUACAUCCG-3′-3′-X | |

where each C, G, A, and U represents, respectively, the naturally-occurring nucleotides cytidine, guanidine, adenine, and uridine, or modified nucleotides corresponding thereto; X-5′-5′ is an inverted nucleotide capping the 5′ terminus of the aptamer; 3′-3′-X is an inverted nucleotide capping the 3′ terminus of the aptamer; and the remaining nucleotides or modified nucleotides are sequentially linked via 5′-3′ phosphodiester linkages. In some embodiments, each of the nucleotides of the capped anti-VEGF aptamer, individually carries a 2′ ribosyl substitution, such as —OH (which is standard for ribonucleic acids (RNAs)), or —H (which is standard for deoxyribonucleic acids (DNAs)). In other embodiments the 2′ ribosyl position is substituted with an O(C₁₋₁₀ alkyl), an O(C₁₋₁₀ alkenyl), a F, an N₃, or an NH₂ substituent.

In one specific embodiment, the 5′-5′ capped anti-VEGF aptamer has the structure:

| | |
|---|---|
| T_{d}-5′-5′-C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}C_{f}Gₘ3′-3′-T_{d} | (SEQ ID NO: 1) |

and where “Gₘ” represents 2′-methoxyguanylic acid, “Aₘ” represents 2′-methoxyadenylic acid, “C_{f}” represents 2′-fluorocytidylic acid, “U_{f}” represents 2′-fluorouridylic acid, “Aᵣ” represents riboadenylic acid, and “T_{d}” represents deoxyribothymidylic acid.

The invention also provides pharmaceutical compositions which includes an effective amount of an aptamer that is capped at the 5′ end with a 5′-5′ inverted nucleoside and at the 3′ end with a 3′-3′ inverted nucleoside, and a pharmaceutically acceptable carrier or diluent. In particular embodiments of this aspect, the capped aptamers may be RNA aptamers, DNA aptamers, or aptamers having a mixed (i.e., both RNA and DNA) composition.

In another particular embodiment, the invention provides pharmaceutical compositions which includes an effective amount of an anti-VEGF aptamer that is capped at the 5′ end with a 5′-5′ inverted nucleoside and at the 3′ end with a 3′-3′ inverted nucleoside, and a pharmaceutically acceptable carrier or diluent. In particular embodiments of this aspect, the capped anti-VEGF aptamers are RNA aptamers, DNA aptamers, or aptamers having a mixed (i.e., both RNA and DNA) composition. In one embodiment, the anti-VEGF capped aptamers of the invention include the nucleotide sequence GAAGAAUUGG (SEQ ID NO: 2). In another embodiment, the anti-VEGF capped aptamers of the invention include the nucleotide sequence UUGGACGC (SEQ ID NO: 3). In still another embodiment of this aspect, the anti-VEGF capped aptamers of the invention include the nucleotide sequence GUGAAUGC (SEQ ID NO: 4). In yet another embodiment, the pharmaceutical compositions of the invention include an anti-VEGF capped aptamer having the sequence:

| | |
|---|---|
| X-5′-5′- | (SEQ ID NO: 1) |
| CGGAAUCAGUGAAUGCUUAUACAUCCG-3′-3′-X | |

where each C, G, A, and U represents, respectively, the naturally-occurring nucleotides cytidine, guanidine, adenine, and uridine, or modified nucleotides corresponding thereto; X-5′-5′ is an inverted nucleotide capping the 5′ terminus of the aptamer; 3′-3′-X is an inverted nucleotide capping the 3′ terminus of the aptamer; and the remaining nucleotides or modified nucleotides are sequentially linked via 5′-3′ phosphodiester linkages. In some embodiments, each of the nucleotides of the capped anti-VEGF aptamer in the pharmaceutical composition, carries a 2′ ribosyl substitution, such as —OH (which is standard for ribonucleic acids (RNAs)), or —H (which is standard for deoxyribonucleic acids (DNAs)). In other embodiments the 2′ ribosyl position is substituted with an O(C₁₋₁₀ alkyl), an O(C₁₋₁₀ alkenyl), a F, an N₃, or an NH₂ substituent. In another specific embodiment, the pharmaceutical composition of the invention includes a capped anti-VEGF aptamer of the invention having the structure:

| | |
|---|---|
| T_{d}-5′-5′-C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}C_{f}Gₘ3′-3′-T_{d} | (SEQ ID NO: 1) |

In this embodiment, “Gₘ” represents 2′-methoxyguanylic acid, “Aₘ” represents 2′-methoxyadenylic acid, “C_{f}” represents 2′-fluorocytidylic acid, “U_{f}” represents 2′-fluorouridylic acid, “Aᵣ” represents riboadenylic acid, and “T_{d}” represents deoxyribothymidylic acid.

In yet another aspect, the invention provides a composition for the sustained release of an aptamer having both 5′-5′ and 3′-3′ capped ends, and a biocompatible polymer that allows for the release of the capped aptamer. In particular embodiments of this aspect, the capped aptamers in the composition for sustained release are RNA aptamers, DNA aptamers, or aptamers having a mixed (i.e., both RNA and DNA) composition.

In another aspect, the invention provides composition for the sustained release of an anti-VEGF aptamer, which includes an anti-VEGF aptamer having both 5′-5′ and 3′-3′ capped ends, and a biocompatible polymer that allows for the release of the capped anti-VEGF aptamer. In particular embodiments of this aspect, the capped anti-VEGF aptamers in the composition for sustained release are RNA aptamers, DNA aptamers, or aptamers having a mixed (i.e., both RNA and DNA) composition. In one embodiment of this aspect, the anti-VEGF capped aptamers of the compositions for sustained release of the invention include the nucleotide sequence GAAGAAUUGG (SEQ ID NO: 2). In another embodiment of this aspect, the anti-VEGF capped aptamers of the invention include the nucleotide sequence UUGGACGC (SEQ ID NO: 3). In still another embodiment of this aspect, the anti-VEGF capped aptamers of the invention include the nucleotide sequence GUGAAUGC (SEQ ID NO: 4). In a particular embodiment, the compositions for sustained release of the invention include an anti-VEGF capped aptamer having the sequence:

| | |
|---|---|
| X-5′-5′- | (SEQ ID NO: 1) |
| CGGAAUCAGUGAAUGCUUAUACAUCCG-3′-3′-X | |

where each C, G, A, and U represents, respectively, the naturally-occurring nucleotides cytidine, guanidine, adenine, and uridine, or modified nucleotides corresponding thereto; X-5′-5′ is an inverted nucleotide capping the 5′ terminus of the aptamer; 3′-3′-X is an inverted nucleotide capping the 3′ terminus of the aptamer; and the remaining nucleotides or modified nucleotides are sequentially linked via 5′-3′ phosphodiester linkages. In some embodiments, each of the nucleotides of the capped anti-VEGF aptamer in the composition for sustained release carries a 2′ ribosyl substitution such as —OH (which is standard for ribonucleic acids (RNAs)), or —H (which is standard for deoxyribonucleic acids (DNAs)). In other embodiments the 2′ ribosyl position is substituted with an O(C₁₋₁₀ alkyl), an O(C₁₋₁₀ alkenyl), a F, an N₃, or an NH₂ substituent. In a particular embodiment, the compositions for sustained release of the invention includes a capped anti-VEGF aptamer of the invention having the structure:

| | |
|---|---|
| T_{d}-5′-5′-C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}C_{f}Gₘ3′-3′-T_{d} | (SEQ ID NO: 1) |

In this embodiment, “Gₘ” represents 2′-methoxyguanylic acid, “Aₘ” represents 2′-methoxyadenylic acid, “C_{f}” represents 2′-fluorocytidylic acid, “U_{f}” represents 2′-fluorouridylic acid, “Aᵣ” represents riboadenylic acid, and “T_{d}” represents deoxyribothymidylic acid.

In one embodiment of this aspect of the invention, the aptamer is present in an amount from about 0.1% (w/w) to about 30% (w/w) of the composition. In other embodiments, the aptamer is present in the composition for sustained release in an amount from about 0.1% (w/w) to about 10% (w/w), or from about 0.5% (w/w) to about 5% (w/w), of the composition. In another embodiment, the composition includes a stabilizing agent such as a saccharide, a poly alcohol, a protein or a hydrophilic polymer. In another embodiment, the biocompatible polymer is a degradable polymer under physiological conditions. In certain embodiments, the degradable polymer is a polycarbonate, a polyanhydride, a polyamide, a polyester, a polyorthoester, a bioerodable hydrogel, a copolymer, or a mixture of two or more of these degradable polymers. In another embodiment, the polyester degradable polymer of the composition for sustained release of the invention is poly(lactic acid), poly(lactic acid-co-glycolic acid), polycaprolactone or a blend or copolymer of one or more of these polyester degradable polymers. In a particular embodiment, the polyester degradable polymer is poly(lactic acid-co-glycolic acid).

In yet another embodiment, the biocompatible polymer utilized for the sustained release of the aptamer is a non-degradable polymer. In some embodiments, the non-degradable polymer is a silicone derivative, or a polysaccharide, a polyether, a vinyl polymer, a polyurethane, a cellulose-based polymer or a polysiloxane. In a particular embodiment, the polyether biocompatible polymer of the composition for sustained release of the invention is a poly(ethylene oxide), a poly(ethylene glycol), or a poly (tetramethylene oxide). In another particular embodiment, the vinyl biocompatible polymer for sustained release of the aptamer is a polyacrylate, an acrylic acid, a poly(vinyl alcohol), a poly (vinyl pyrolidone) or a poly(vinyl acetate). In yet another embodiment, the biocompatible polymer is a cellulose-based polymer such as cellulose, alkyl cellulose, hydroxyalkyl cellulose, cellulose ethers, cellulose esters, nitrocellulose, or cellulose acetate.

In still another embodiment, the composition for sustained release of the anti-VEGF aptamer includes a microsphere. In one embodiment, the microsphere comprises a biocompatible polymer. In another embodiment, the composition for sustained release of the invention includes a solid particulate having an average diameter of less than about 400 μm. In other embodiments, the microsphere is a solid particulate having an average diameter of less than about 200 μm or less than about 100 μm.

In yet another aspect, the invention provides compositions for the sustained release of an anti-VEGF aptamer having both 5′-5′ and 3′-3′ capped that include a biocompatible polymer which is degradable under physiological conditions. In one embodiment of this aspect, the half-life for the release of the capped anti-VEGF aptamer from the degradable biocompatible polymer while on the sclera of an eye is greater than about one month. In other embodiments, the half-life for the release of the capped anti-VEGF aptamer from the degradable biocompatible polymer while on the sclera of an eye is greater than about two months, or greater than about four months.

In another aspect, the invention provides a method of treating or inhibiting an ocular disease state in a mammal by administering to the mammal, in an amount sufficient to treat or inhibit the ocular disease, an anti-VEGF aptamer having 5′-5′ and 3′-3′ inverted caps. In a particular embodiment of this aspect, the method of the invention includes administering the anti-VEGF aptamer having 5′-5′ and 3′-3′ inverted caps together with a pharmaceutically acceptable carrier or diluent.

In some embodiments of this method, the anti-VEGF capped aptamers are RNA aptamers, DNA aptamers, or aptamers having a mixed (i.e., both RNA and DNA) composition.

In another embodiment of this method the anti-VEGF capped aptamers administered with the pharmaceutically acceptable carrier or diluent include the nucleotide sequence GAAGAAUUGG (SEQ ID NO: 2). In another embodiment, the anti-VEGF capped aptamers administered include the nucleotide sequence UUGGACGC (SEQ ID NO: 3). In still another embodiment, the anti-VEGF capped aptamers administered include the nucleotide sequence GUGAAUGC (SEQ ID NO: 4).

In one embodiment of this method the anti-VEGF capped aptamers administered have the sequence:

| | |
|---|---|
| X-5′-5′- | (SEQ ID NO: 1) |
| CGGAAUCAGUGAAUGCUUAUACAUCCG-3′-3′-X | |

where each C, G, A, and U represents, respectively, the naturally-occurring nucleotides cytidine, guanidine, adenine, and uridine, or modified nucleotides corresponding thereto; X-5′-5′ is an inverted nucleotide capping the 5′ terminus of the aptamer; 3′-3′-X is an inverted nucleotide capping the 3′ terminus of the aptamer; and the remaining nucleotides or modified nucleotides are sequentially linked via 5′-3′ phosphodiester linkages. In some embodiments, each of the nucleotides of the capped anti-VEGF aptamer carries a 2′ ribosyl substitution such as —OH (which is standard for ribonucleic acids (RNAs)), or —H (which is standard for deoxyribonucleic acids (DNAs)). In other embodiments the 2′ ribosyl position is substituted with an O(C₁₋₁₀ alkyl), an O(C₁₋₁₀ alkenyl), a F, an N₃, or an NH₂ substituent.

In one particular embodiment of this method, the anti-VEGF capped aptamers administered have the structure:

| | |
|---|---|
| T_{d}-5′-5′-C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}C_{f}Gₘ3′-3′-T_{d} | (SEQ ID NO: 1) |

In this embodiment, “Gₘ” represents 2′-methoxyguanylic acid; “Aₘ” represents 2′-methoxyadenylic acid; “C_{f}” represents 2′-fluorocytidylic acid; “U_{f}” represents 2′-fluorouridylic acid; “Aᵣ” represents riboadenylic acid; and “T_{d}” represents deoxyribothymidylic acid.

The invention also provides another method of treating or inhibiting an ocular disease state in a mammal by administering to the mammal, in an amount sufficient to treat or inhibit the ocular disease, an anti-VEGF aptamer having 5′-5′ and 3′-3′ inverted caps. In this aspect, the method includes administering the effective amount of anti-VEGF aptamer having 5′-5′ and 3′-3′ inverted caps together with a biocompatible polymer that allows for the sustained release of the aptamer.

In some embodiments of this method, the anti-VEGF capped aptamers administered with the biocompatible polymer that allows for the sustained release of the aptamer are RNA aptamers or DNA aptamers. In still other embodiments the capped anti-VEGF aptamers administered have a mixed (i.e., both RNA and DNA) composition.

In another embodiment of this method, the anti-VEGF capped aptamers include the nucleotide sequence GAAGAAUUGG (SEQ ID NO: 2). In another embodiment, the anti-VEGF capped aptamers administered include the nucleotide sequence UUGGACGC (SEQ ID NO: 3). In still another embodiment, the anti-VEGF capped aptamers administered include the nucleotide sequence GUGAAUGC (SEQ ID NO: 4).

In one particular embodiment of this method, the anti-VEGF capped aptamers administered with the biocompatible polymer that allows for the sustained release of the aptamer have the sequence:

| | |
|---|---|
| X-5′-5′- | (SEQ ID NO: 1) |
| CGGAAUCAGUGAAUGCUUAUACAUCCG-3′-3′-X | |

where each C, G, A, and U represents, respectively, the naturally-occurring nucleotides cytidine, guanidine, adenine, and uridine, or modified nucleotides corresponding thereto; X-5′-5′ is an inverted nucleotide capping the 5′ terminus of the aptamer; 3′-3′-X is an inverted nucleotide capping the 3′ terminus of the aptamer; and the remaining nucleotides or modified nucleotides are sequentially linked via 5′-3′ phosphodiester linkages. In some embodiments, each of the nucleotides of the capped anti-VEGF aptamer that is administered with the biocompatible polymer carries a 2′ ribosyl substitution, such as —OH (which is standard for ribonucleic acids (RNAs)), or —H (which is standard for deoxyribonucleic acids (DNAs)). In other embodiments the 2′ ribosyl position is substituted with an O(C₁₋₁₀ alkyl), an O(C₁₋₁₀ alkenyl), a F, an N₃, or an NH₂ substituent.

In one specific embodiment, the anti-VEGF capped aptamers administered with the biocompatible polymer have the structure:

| | |
|---|---|
| T_{d}-5′-5′-C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}C_{f}Gₘ3′-3′-T_{d} | (SEQ ID NO: 1) |

In this embodiment, “Gₘ” represents 2′-methoxyguanylic acid, “Aₘ” represents 2′-methoxyadenylic acid, “C_{f}” represents 2′-fluorocytidylic acid, “U_{f}” represents 2′-fluorouridylic acid, “Aᵣ” represents riboadenylic acid, and “T_{d}” represents deoxyribothymidylic acid.

In another embodiment of this aspect, the composition for sustained release administered includes a 5′-5′ and 3′-3′capped anti-VEGF aptamer present at a concentration of about 0.1% (w/w) to about 30% (w/w) of the composition for sustained release. In another embodiment, the composition for sustained release administered includes an anti-VEGF aptamer that is about 0.1% (w/w) to about 10% (w/w) of the composition. In still another embodiment, the composition for sustained release administered includes an anti-VEGF aptamer that is about 0.5% (w/w) to about 5% (w/w) of the composition.

In yet another embodiment, the composition for sustained release includes a stabilizing agent such as a saccharide, a polyalcohol, a protein or a hydrophilic polymer.

In some embodiments, the biocompatible polymer administered in combination with the anti-VEGF aptamer is degradable under physiological conditions. In some embodiments, the degradable polymers for administration in combination with the capped anti-VEGF aptamers to treat ocular disease include polycarbonates, polyanhydrides, polyamides, polyesters, polyorthoesters, bioerodable hydrogels, as well as copolymers, and mixtures thereof.

In particular embodiments, the degradable biopolymer administered in the composition for sustained release is a polyester such as a poly(lactic acid), a poly(glycolic acid), a poly(lactic acid-co-glycolic acid), a polycaprolactone, or a blend or copolymer thereof. In a particularly useful embodiment, the degradable biopolymer includes poly(lactic acid-co-glycolic acid).

In other embodiments of this aspect of the invention, the biocompatible polymer is a non-degradable polymer, such as a silicone derivative. In other embodiments, the non-degradable polymer administered in the composition for sustained release is a polysaccharide, a polyether, a vinyl polymer, a polyurethane, a cellulose-based polymer, or a polysiloxane. In a particular embodiment, the polyether non-degradable biocompatible polymer administered with the composition for sustained release is a poly (ethylene oxide), a poly (ethylene glycol), or a poly (tetramethylene oxide). In another particular embodiment, the vinyl polymer non-degradable biocompatible polymer administered with the composition for sustained release is a polyacrylates, an acrylic acid, a poly (vinyl alcohol), a poly (vinyl pyrolidone) or a poly (vinyl acetate). In yet another particular embodiment, the cellulose-based non-degradable biocompatible polymer administered with the composition for sustained release is cellulose, alkyl cellulose, hydroxyalkyl cellulose, a cellulose ether, a cellulose ester, nitrocellulose, or a cellulose acetate.

In a particular embodiment, the composition for sustained release that is administered is a solid particulate with an average diameter of less than about 400 μm. In other embodiments, the administered solid particulate has an average diameter of less than about 200 μm or less than about 100 μm.

In still other embodiments of this aspect of the invention, the biocompatible polymer is degradable under physiological conditions. In certain embodiments, the half-life for the release of the anti-VEGF aptamer on the sclera of an eye is greater than about one month. In other embodiments, the half-life for the release of the aptamer on the sclera of an eye is greater than about two months. In another embodiment, the half-life for the release of the aptamer on the sclera of an eye is greater than about four months.

In certain embodiments of this aspect of the invention, the disease state treated or inhibited is optic disc neovascularization, iris neovascularization, retinal neovascularization, choroidal neovascularization, corneal neovascularization, intravitreal neovascularization, glaucoma, pannus, pterygium, macular edema, diabetic macular edema, vascular retinopathy, retinal degeneration, uveitis, inflammatory diseases of the retina, or proliferative vitreoretinopathy. In some embodiments, the corneal neovascularization to be treated or inhibited is caused by trauma, chemical burns or corneal transplantation. In other particular embodiments, the iris neovascularization to be treated or inhibited is caused by diabetic retinopathy, vein occlusion, ocular tumor or retinal detachment. In still other particular embodiments, the retinal neovascularization to be treated or inhibited is caused by diabetic retinopathy, vein occlusion, sickle cell retinopathy, retinopathy of prematurity, retinal detachment, ocular ischemia or trauma. In yet other particular embodiments, the intravitreal neovascularization to be treated or inhibited is caused by diabetic retinopathy, vein occlusion, sickle cell retinopathy, retinopathy of prematurity, retinal detachment, ocular ischemia or trauma.

In further embodiments, the choroidal neovascularization to be treated or inhibited is caused by retinal or subretinal disorders of age-related macular degeneration, diabetic macular edema, presumed ocular histoplasmosis syndrome, myopic degeneration, angioid streaks or ocular trauma.

In another embodiment of the invention, administration of the therapeutic agent is achieved by placing the composition in contact with the sclera of the eye of the mammal via drops or drug delivery devices. Suitable drug delivery devices for administration of the therapeutic agents of the invention include micromechanical drug delivery systems that are implanted inside the human eye socket directly onto the white surface (sclera) of the eye, e.g., that described in co-pending U.S. patent application Ser. No. 10/139,656. In yet other embodiments, administration of the therapeutic agent is achieved by intravitreal injection, subconjunctival injection or subconjunctival administration.

### BRIEF DESCRIPTION OF THE DRAWINGS

FIG. 1 is a schematic representation of the chemical structure of a 5′-5′ inverted cap.

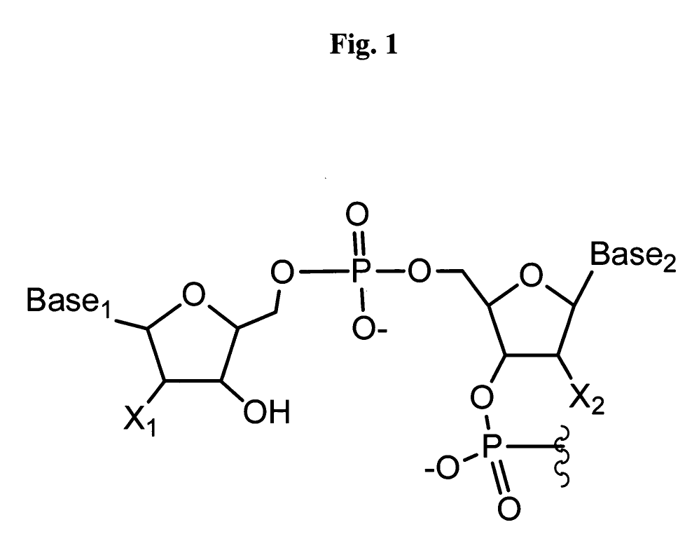

FIG. 2 is a schematic representation of the chemical structure of a 3′-3′ inverted cap.

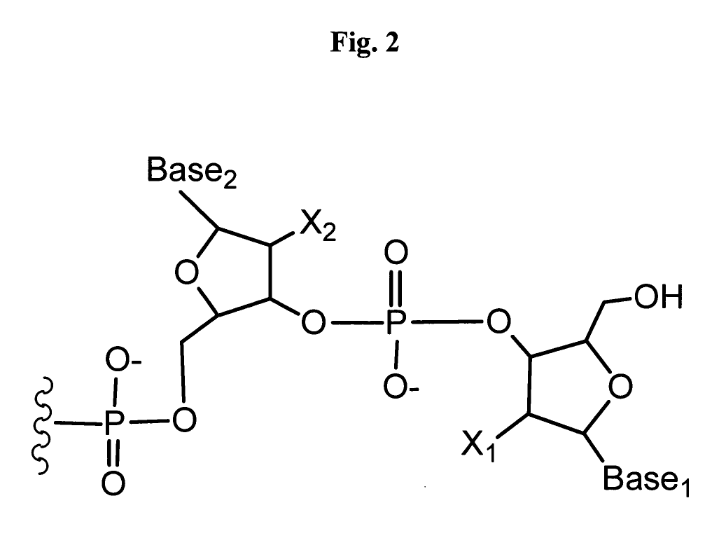

FIG. 3 is a schematic representation of the secondary structure of a 5′-5′ inverted dT and 3′-3′ inverted dT capped anti-VEGF aptamer as established by one and two dimensional proton NMR spectroscopy.

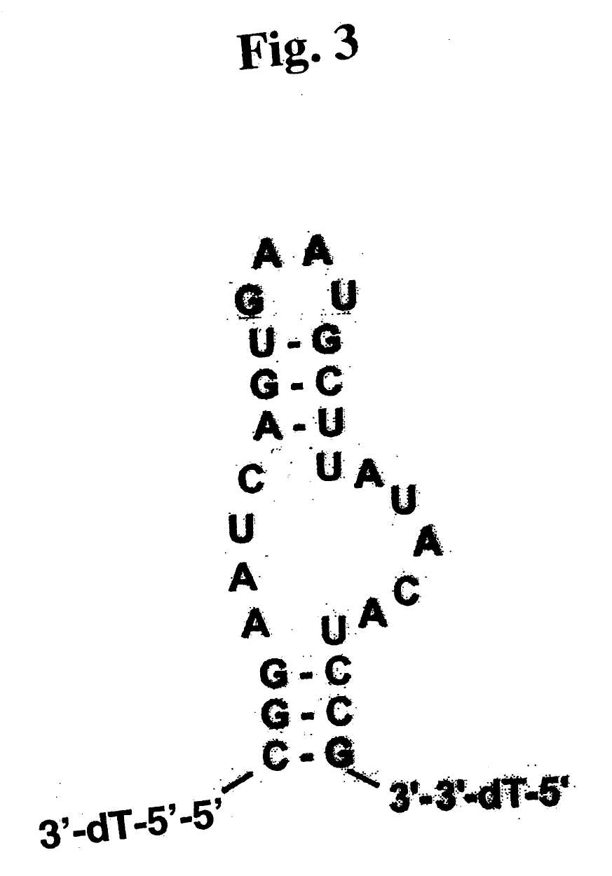

FIG. 4 is a diagrammatic representation of multiple VEGF-A isoforms (VEGF₁₂₁, VEGF₁₄₅, VEGF₁₆₅, VEGF₁₈₃, and VEGF₁₈₉) with varying functions.

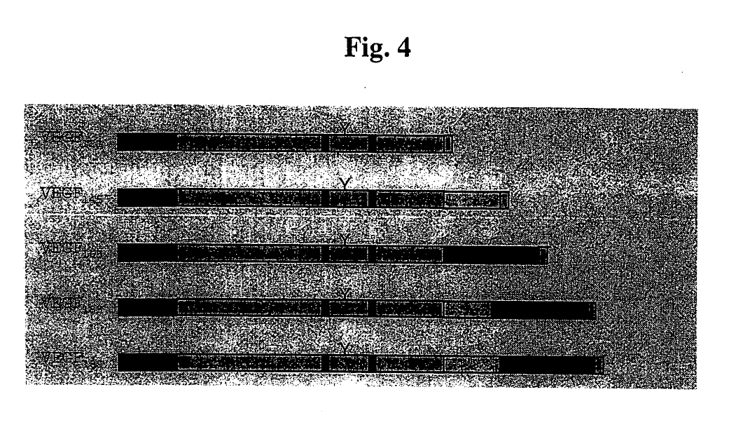

### DETAILED DESCRIPTION OF THE INVENTION

The patent, scientific and medical publications referred to herein establish knowledge that was available to those of ordinary skill in the art at the time the invention was made. The entire disclosures of the issued U.S. patents, published and pending U.S. patent applications, published PCT international patent applications and other references cited herein are hereby incorporated by reference.

Definitions

All technical and scientific terms used herein, unless otherwise defined below, are intended to have the same meaning as commonly understood by one of ordinary skill in the art; references to techniques employed herein are intended to refer to the techniques as commonly understood in the art, including variations on those techniques or substitutions of equivalent or later-developed techniques which would be apparent to one of skill in the art. In order to more clearly and concisely describe the subject matter which is the invention, the following definitions are provided for certain terms which are used in the specification and appended claims.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the term “aptamer” means any polynucleotide, or salt thereof, having selective binding affinity for a non-polynucleotide molecule (such as a protein) via non-covalent physical interactions. An aptamer is a polynucleotide that binds to a ligand in a manner analogous to the binding of an antibody to its epitope. Aptamers of the invention are modified as described herein by incorporating 5′-5′ and 3′-3′ inverted caps in the sequence.

The terms “polynucleotide” and “oligonucleotide” are meant to encompass any molecule comprising a sequence of covalently joined nucleosides or modified nucleosides which has selective binding affinity for a naturally-occurring nucleic acid of complementary or substantially complementary sequence under appropriate conditions (e.g., pH, temperature, solvent, ionic strength, electric field strength). Polynucleotides include naturally-occurring nucleic acids as well as nucleic acid analogues with modified nucleosides or internucleoside linkages, and molecules which have been modified with linkers or detectable labels which facilitate conjugation or detection.

As used herein, the term “nucleoside” means any of the naturally occurring ribonucleosides or deoxyribonucleosides: adenosine, cytosine, guanosine, thymosine or uracil.

The term “modified nucleotide” or “modified nucleoside” or “modified base” refer to variations of the standard bases, sugars and/or phosphate backbone chemical structures occurring in ribonucleic (i.e., A, C, G and U) and deoxyribonucleic (i.e., A, C, G and T) acids. For example, Gₘ represents 2′-methoxyguanylic acid, Aₘ represents 2′-methoxyadenylic acid, C_{f} represents 2′-fluorocytidylic acid, U_{f} represents 2′-fluorouridylic acid, Aᵣ, represents riboadenylic acid. The aptamer includes cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer further includes guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer further includes adenine or any adenine-related base including 6-methyladenine, N6-isopentenyladenine, N6-methyladenine, 1-methyladenine, 2-methyladenine, 2-methylthio-N-6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included is uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil. Examples of other modified base variants known in the art include, without limitation, those listed at 37 C.F.R. §1.822(p) (1), e.g., 4-acetylcytidine, 5-(carboxyhydroxylmethyl)uridine, 2′-methoxycytidine, 5-carboxymethylaminomethyl-2-thioridine, 5-carboxymethylaminomethyluridine, dihydrouridine, 2′-O-methylpseudouridine, β-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, β-D-mannosylqueosine, 5-methoxycarbonylmethyluridine, 5-methoxyuridine, 2-methylthio-N-6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-β-D-ribofuiranosylpurine-6-yl)carbamoyl)threonine, 2′-O-methyl-5-methyluridine, 2′-O-methyluridine, wybutosine, 3-(3-amino-3-carboxypropyl)uridine. Nucleotides also include any of the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941. Examples of modified nucleoside and nucleotide sugar backbone variants known in the art include, without limitation, those having, e.g., 2′ ribosyl substituents such as F, SH, SCH₃, OCN, Cl, Br, CN, CF₃, OCF₃, SOCH₃, SO₂, CH₃, ONO₂, NO₂, N₃, NH₂, OCH₂CH₂OCH₃, O(CH₂)₂ON(CH₃)₂, OCH₂OCH₂N(CH₃)₂, O(C₁₋₁₀ alkyl), O(C₂₋₁₀ alkenyl), O(C₂₋₁₀ alkynyl), S(C₁₋₁₀-alkyl), S(C₂₋₁₀ alkenyl), S(C₂₋₁₀ alkynyl), NH(C₁₋₁₀ alkyl), NH(C₂₋₁₀ alkenyl), NH(C₂₋₁₀ alkynyl), and O-alkyl-O-alkyl. Desirable 2′ ribosyl substituents include 2′-methoxy (2′-OCH₃), 2′-aminopropoxy (2′ OCH₂CH₂CH₂NH₂), 2′-allyl (2′-CH₂—CH═CH₂), 2′-O-allyl (2′-O—CH₂—CH═CH₂), 2′-amino (2′-NH₂), and 2′-fluoro (2′-F). The 2′-substituent may be in the arabino (up) position or ribo (down) position.

As used herein, the term “5′-5′ inverted nucleotide cap” means a first nucleotide covalently linked to the 5′ end of an oligonucleotide via a phosphodiester linkage between the 5′ position of the first nucleotide and the 5′ terminus of the oligonucleotide as shown below.

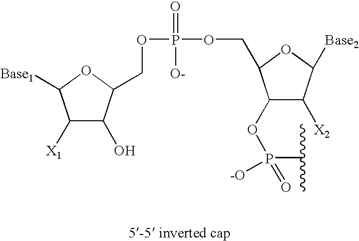

The term “3′-3′ inverted nucleotide cap” is used herein to mean a last nucleotide covalently linked to the 3′ end of an oligonucleotide via a phosphodiester linkage between the 3′ position of the last nucleotide and the 3′ terminus of the oligonucleotide as shown below.

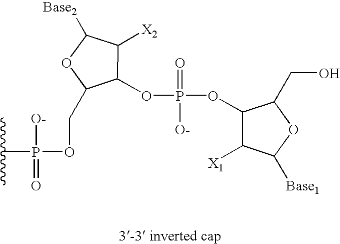

“Anti-VEGF aptamers,” are meant to encompass polynucleotide aptamers that bind to, and inhibit the activity of, VEGF. Such aptamers can be identified using known methods. For example, Systematic Evolution of Ligands by Exponential enrichment, or SELEX, methods can be used as described in U.S. Pat. Nos. 5,475,096 and 5,270,163. Anti-VEGF aptamers include the sequences described in U.S. Pat. Nos. 6,168,778, 6,051,698, 5,859,228, and 6,426,335, which can be modified, in accordance with the present invention, to include both 5′-5′ and 3′-3′ inverted caps.

Unless specifically indicated otherwise, the word “or” is used herein in the inclusive sense of “and/or” and not the exclusive sense of “either/or.”

As used herein, the terms “increase” and “decrease” mean, respectively, a statistically significantly increase (i.e., p<0.1) and a statistically significantly decrease (i.e., p<0.1).

The recitation of a numerical range for a variable, as used herein, is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2, can be 0, 1 or 2 for variables which are inherently discrete, and can be 0.0, 0.1, 0.01, 0.001, or any other real value ≦0 and <2 for variables which are inherently continuous.

5′ and 3′-Capped Aptamers

Aptamers have been made which are stable in the presence of nucleases. Such aptamers include a 5′-5′ inverted nucleotide cap at the 5′ terminus of the aptamer and a 3′-3′ inverted nucleotide cap at the 3′ terminus of the aptamer. These structural modifications in the 5′ and 3′ ends serve to stabilize the aptamer compounds of the invention. Aptamers modified in this manner are useful for the treatment of diseases associated with a protein target to which the aptamer binds.

The invention features 5′-5′ and 3′-3′ inverted nucleotide capped aptamers that are composed of RNA, DNA or RNA and DNA. Examples of useful aptamers are aptamers that can be used for treating neovascularization such as in disease states resulting from unwanted VEGF-induced vascularization, particularly diseases of the eye.

Particular non-limiting compositions of the invention include aptamers which contain the sequence GAAGAAUUGG (SEQ ID NO: 2), UUGGACGC (SEQ ID NO: 3), or GUGAAUGC (SEQ ID NO: 4), wherein each C, G, A or U is a nucleotide or modified nucleotide as defined above.

The invention includes 5′-5′ and 3′-3′ capped anti-VEGF aptamers containing the sequence GAAGAAUUGG (SEQ ID NO: 2), UUGGACGC (SEQ ID NO: 3), or GUGAAUGC (SEQ ID NO: 4), wherein each C, G, A or U is a nucleotide or modified nucleotide as defined above. For example, the invention includes 5′-5′ and 3′-3′ capped anti-VEGF aptamers including subsequences corresponding to SEQ ID NOS: 2-3, such as the products of Systematic Evolution of Ligands by Exponential enrichment (SELEX) described in, e.g., U.S. Pat. Nos. 6,426,335, 6,168,778, 6,147,204, 6,051,698, 6,011,020, 5,958,691, 5,859,228, 5,849,479 and 5,811,533, the contents of which are incorporated herein in their entirety. Nonlimiting and exemplary anti-VEGF aptamer sequences comprising the subsequence GAAGAAUUGG (SEQ ID NO: 2) include: UAGGAAGAAUUGGAAGCGCAUUUUCCUCG (SEQ ID NO: 5) and AACGGAAGAAUUGGAUACGUAGCAUGCGU (SEQ ID NO: 6). Nonlimiting and exemplary anti-VEGF aptamers sequences comprising the subsequence UUGGACGC (SEQ ID NO: 3) include: GAACCGAUGGAAUUUUUGGACGCUCGCCU (SEQ ID NO: 7) and UAACCGAAUUGAAGUUAUUGGACGCUACCU (SEQ ID NO: 8). Nonlimiting and exemplary anti-VEGF aptamers sequences comprising the subsequence GUGAAUGC (SEQ ID NO: 4) include: AGAAUCAGUGAAUGCUUAUAAAUCUCGCGU (SEQ ID NO: 9) and AAUCAGUGAAUGCUUAUACAUCCGCUCGGU (SEQ ID NO: 10).

Still other anti-VEGF aptamer sequences include alternative high-affinity sequences known in the art, e.g., single-nucleotide and multiple nucleotide substitutions of these and other anti-VEGF aptamers that bind to VEGF with comparable affinity. For example, the invention includes aptamer nucleic acid sequences that are substantially homologous to and that have substantially the same ability to bind VEGF as the specific aptamer sequence shown herein, e.g., those specified by SEQ ID NOS: 1-10. By “substantially homologous” it is meant a degree of primary sequence homology in excess of 70%, such as in excess of 80%, in excess of 90%, 95%, or 99%. The percentage of homology as described herein is calculated as the percentage of nucleotides found in the smaller of the two sequences that align with identical nucleotide residues in the sequence being compared when one gap in a length of 10 nucleotides may be introduced to assist in that alignment. The percent homology, or sequence identity, of such related sequences may also be determined using known algorithms, e.g., through the BLAST network service (Altschul, S. F. et al., (1990) J. Mol. Biol. 215: 403-410) provided by the National Center for Biotechnology Information. Substantially the same ability to bind VEGF means that the affinity is within one or two orders of magnitude of the affinity of the ligands described herein. It is well within the skill of those of ordinary skill in the art to determine whether a given sequence, substantially homologous to those specifically described herein, has the same ability to bind VEGF.

Still other aptamer sequences with the same structure or structural motifs as postulated by sequence alignment using, e.g., the Zukerfold program (see Zuker (1989) Science 244: 48-52), are included. As known in the art, other computer programs can be used to predict secondary structure and structural motifs. Substantially the same structure or structural motifs of aptamers in solution or as a bound aptamer/VEGF complex can also be postulated using NMR or other techniques as would be known in the art. U.S. Pat. No. 6,344,318, the contents of which are incorporated herein by reference, describes methods for identifying such structurally related aptamers of the invention.

Also included within the invention are modified aptamers, having improved properties such as decreased size, enhanced stability, or enhanced binding affinity. Such modifications of the anti-VEGF aptamer sequences include adding, deleting or substituting nucleotide residues, and/or chemically modifying one or more residues. Methods for producing such modified anti-VEGF aptamers are known in the art and described in, e.g., U.S. Pat. Nos. 5,817,785 and 5,958,691.

For example, chemically modified aptamers include those containing one or more modified bases. For example, the modified pyrimidine bases of the present invention may have substitutions of the general formula 5′-X and/or 2′-Y, and the modified purine bases may have modifications of the general formula 8-X and/or 2-Y. The group X includes the halogens I, Br, Cl, or an azide or amino group. The group Y includes an amino group, fluorine, or a methoxy group. Other functional substitutions that would serve the same function may also be included. The aptamers of the present invention may have one or more X-modified bases, or one or more Y-modified bases, or a combination of X- and Y-modified bases. The present invention encompasses derivatives of these substituted pyrimidines and purines such as 5′-triphosphates, and 5′-dimethoxytrityl, 3′-beta-cyanoethyl, N,N-diisopropyl phosphoramidites with isobutyryl protected bases in the case of adenosine and guanosine, or acyl protection in the case of cytosine. Further included in the present invention are aptamers bearing any of the nucleotide analogs herein disclosed. The present invention encompasses specific nucleotide analogs modified at the 5 and 2′ positions, including 5-(3-aminoallyl)uridine triphosphate (5-AA-UTP), 5-(3-aminoallyl)deoxyuridine triphosphate (5-AA-dUTP), 5-fluorescein-12-uridine triphosphate (5-F-12-UTP), 5-digoxygenin-11-uridine triphosphate (5-Dig-11-UTP), 5-bromouridine triphosphate (5-Br-UTP), 2′-amino-uridine triphosphate (2′-NH.sub.2-UTP) and 2′-amino-cytidine triphosphate (2′-NH.sub.2-CTP), 2′-fluoro-cytidine triphosphate (2′-F-CTP), and 2′-fluoro-uridine triphosphate (2′-F-UTP).

Some aptamers of the invention have the following formula I:

| | |
|---|---|
| X-5′-5′-CGGAAUCAGUGAAUGCUUAUACAUCCG- | (SEQ ID NO:1) |
| 3′-3′-X | |

wherein C, G, A, and U represent their respective cytidylic, guanylic, adenylic, and uridylic acid nucleotides, X-5′-5′ is an inverted nucleotide capping the 5′ terminus of the aptamer and 3′-3′-X is an inverted nucleotide capping the 3′ terminus of the aptamer, and the remaining nucleotides are sequentially linked via 5′-3′ phosphodiester linkages. In formula I, each of the nucleotides may, individually, include a 2′ ribosyl substituent selected from OH, H, O(C₁₋₁₀ alkyl), O(C₁₋₁₀ alkenyl), F, N₃, and NH₂.

Other aptamers of the invention have the following formula II:

| | |
|---|---|
| Td-5′-5′-C_{f}GₘGₘAᵣAᵣU_{f}C_{f}AₘGₘU_{f}GₘAₘAₘU_{f} | (SEQ ID NO:1) |
| GₘC_{f}U_{f}U_{f}AₘU_{f}AₘC_{f}AₘU_{f}C_{f}C_{f}Gₘ-3′-3′-Td | |

In formula II, Gₘ represents 2′-methoxyguanylic acid; Aₘ represents 2′-methoxyadenylic acid; C_{f} represents 2′-fluorocytidylic acid; U_{f} represents 2′-fluorouridylic acid; Aᵣ, represents riboadenylic acid; and T_{d} represents deoxyribothymidylic acid.
Aptamer Synthesis

The anti-VEGF aptamers described herein can be prepared using an automated synthesizer, e.g., standard solid-phase phosphoramidite techniques, as described in Example 1 (see, for example, Scaringe et al. (1990) Nucleic Acids Res. 18:5433 or Wincott et al. (1995) Nucleic Acids Res. 23:2677). The first component employed for the solid-phase synthesis of the aptamers described herein can be, for example, a functionalized support resin including a first nucleoside monomer attached to the resin via its 5′ position to yield the requisite 3′-3′ cap upon subsequent coupling of 3′-phosphoramidite nucleosides to form an oligonucleotide. Support resins for the preparation of this component are known in the art, e.g. as described in Atkinson and Smitt in Oligonucleotide Synthesis (1984) M. J. Gait (ed). 35-49. The last component employed in the solid-phase synthesis can be, e.g., a 5′-phosphoramidite nucleoside, yielding the requisite 5′-5′ inverted cap in the aptamer. Generally the last chain member added to yield the requisite 5′-5′ inverted cap in the aptamer is a 5′-activated and 3′-protected nucleoside, such as a 5′-phosphorous ester amide or a nucleoside H-phosphonate, protected on the 3′-OH group, such as by using dimethoxytrityl. Such 5′-activated nucleosides are known in the art and are available commercially, e.g., dT-5′-CE phosphoramidite shown below (i.e., 3′-dimethoxytrityl-2′-deoxythymidine, 5′-[(2-cyanoethyl)-(N,N-diisopropyl)]-phosphoramidite) available from Glen Research (catalog # 10-0301-10; www.glenresearch.com). 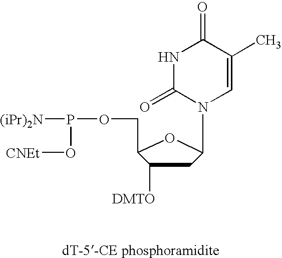
Other 5′-activated and 3′-protected nucleosides corresponding to, e.g., dA, dC, dG, and dU are also available for use in preparing the inverted 5′-5′ cap structure. The corresponding 5′-activated and 3′-protected nucleosides may also be used to form the inverted 5′-5′ aptamer cap.

The aptamers described herein can also be made using other routine methods (see, for example, Methods in Molecular Biology, Volume 20: Protocols for Oligonucleotides and Analogs, pp. 165-189 (S. Agrawal, Ed., Humana Press, 1993); Oligonucleotides and Analogues: A Practical Approach pp. 87-108 (F. Eckstein, Ed., IRL Press, 1991); and Methods in Molecular Biology Volume 74: Ribozyme Protocols, pp. 59-68, Wincott and Usman, “A Practical Method for the Production of RNA and Ribozymes” (P. Turner, Ed., Humana Press, 1997).

In general, the assembly and isolation of the aptamer oligonucleotide involves four steps: synthesis, cleavage and deprotection, desilylation and precipitation. Synthesis of the aptamer may be effected by standard solid phase synthesis procedures using an oligonucleotide synthesizer. The synthesis cycle consists of four steps: (1) removal of the trityl group on the growing chain of the support-bound oligonucleotide (e.g., with dichloroacetic acid (DCA) in dichloromethane (DCM)); (2) activator-mediated coupling of the incoming amidite to the growing chain (e.g., using 4,5-dicyanoimidazole (DCI)-activated coupling); (3) oxidation of the phosphate triester linkage formed in the coupling to a phosphate linkage, and (4) capping of any unreacted growing chain to prevent the formation of deletion sequences. This series of four reactions begins with the inverted thymidine-solid support (e.g., a controlled pore glass (CPG) solid support) and is repeated in an iterative fashion until the aptamer of correct sequence is assembled. The oligonucleotide is then cleaved from its solid support and the base and backbone protecting groups are removed under basic conditions (e.g., using a mixture of methylamine and concentrated ammonia). The two silyl protecting groups on the ribose residues are then removed in a fluoride-mediated reaction (e.g., with hydrogen fluoride or tetrabutylammonium fluoride). Finally, the full deprotected oligonucleotide is isolated (e.g., by chromatography or by precipitation using, e.g., sodium chloride and ethanol).

It is understood that alternative synthetic schemes known in the art are included in the invention. For example, Scaringe et al. ((1998) J. Am. Chem. Soc. 120: 11820-11821) describes an oligonucleotide synthetic scheme that is particularly useful in making RNA and mixed RNA/DNA oligonucleotides. In brief, the method uses silyl ethers for the protection of the 5′-hydroxyl and acid-labile orthoesters for the protection of the 2′-hydroxyl group, i.e., using 5′-O-SIL-2′-O-bis(2-acetoxyethoxy)methyl ribonucleoside phosphoroamidites. The silyl ether protecting groups can be removed with fluoride ions under neutral conditions that are compatible with an acid labile 2′-hydroxyl moiety. The 2′-O-bis(2-acetoxyethoxy)methyl (ACE) orthoester is stable to nucleoside and oligonucleotide synthesis conditions but is modified via ester hydrolysis during base deprotection of the oligonucleotide. The resulting 2′-O-bis(2-hydroxyethodxy)methyl orthoester is ten times more acid-labile than the ACE orthoester and complete cleavage of the 2′-O-protecting groups is effected using extremely mild condition (e.g., using 10 minutes at pH3, 55° C.). The novel features of this chemistry enable the synthesis of RNA oligonucleotides of high quality.

The aptamers can be used, in therapeutic amounts, to treat or inhibit an ocular disease state in a mammal, e.g., a human. The ocular disease state to be treated can be optic disc neovascularization, iris neovascularization, retinal neovascularization, choroidal neovascularization, corneal neovascularization, vitreal neovascularization, glaucoma, pannus, pterygium, macular edema, diabetic macular edema, vascular retinopathy, retinal degeneration, uveitis, inflammatory diseases of the retina, and proliferative vitreoretinopathy. The corneal neovascularization to be treated or inhibited can be caused by trauma, chemical burns or corneal transplantation. The iris neovascularization to be treated or inhibited can be caused by diabetic retinopathy, vein occlusion, ocular tumor or retinal detachment. The retinal neovascularization to be treated or inhibited can be caused by diabetic retinopathy, vein occlusion, sickle cell retinopathy, retinopathy of prematurity, retinal detachment, ocular ischemia or trauma. The intravitreal neovascularization to be treated or inhibited can be caused by diabetic retinopathy, vein occlusion, sickle cell retinopathy, retinopathy of prematurity, retinal detachment, ocular ischemia or trauma. The choroidal neovascularization to be treated or inhibited can be caused by retinal or subretinal disorders of age-related macular degeneration, diabetic macular edema, presumed ocular histoplasmosis syndrome, myopic degeneration, angioid streaks or ocular trauma.

The amount of aptamer administered in any particular case will depend on the disease being treated, mode of administration, and the age, body weight, and general health of the subject. Standard clinical trials may be used to determine effective doses and optimal dosing regimens.

The inverted cap anti-VEGF aptamers of the invention can be used to treat or inhibit any ocular disease state involving unwanted neovascularization. The aptamer, in a suitable therapeutic formulation (see below), may be administered by any appropriate route for treatment or inhibition of an ocular disease state. The aptamers may be administered to humans, domestic pets, livestock, or other mammals. Administration to the eye may be, for example, transcleral, subconjunctival, sub-tenon, retro-bulbar or by intravitreous injection.

The aptamers of the invention can also be used to treat non-ocular disease states involving unwanted VEGF-induced neovascularization. Examples are atheroma, Kaposi's sarcoma, haemangioma, collagen vascular diseases, psoriasis, cerebral edema and neoplastic diseases (cancer). Administration of aptamers to treat these disease states can be by any suitable route, including topical, oral, intravenous, subcutaneous, or intravascular administration.

Formulation of 5′-5′,3′-3′ Inverted Cap Aptamers

The aptamers are administered together with any suitable pharmaceutically acceptable carrier or excipient, e.g., saline or distilled water. Optionally, the formulations described herein include excipients that stabilize the aptamer, thereby maintaining therapeutic activity. Furthermore, excipients such as salts, sugars and alcohols, facilitate diffusion of the aptamer therapeutic. Non-limiting representative excipients that can be used in combination with the present invention include saccharides, such as sucrose, trehalose, lactose, fructose, galactose, mannitol, dextran and glucose; poly alcohols, such as glycerol or sorbitol; proteins, such as albumin; hydrophobic molecules, such as oils; and hydrophilic polymers, such as polyethylene glycol, among others. Pharmaceutical formulations of compounds of the invention described herein includes isomers such as diastereomers and enantiomers, mixtures of isomers, including racemic mixtures, salts, solvates, and polymorphs thereof.

Therapeutic formulations may be in the form of liquid solutions or suspensions. Methods well known in the art for making formulations are found, for example, in Remington: The Science and Practice of Pharmacy (20th ed., ed. A. R. Gennaro AR.), Lippincott Williams & Wilkins, 2000. For oral administration, formulations may be in the form of tablets or capsules. Intranasal formulations may be in the form of powders, nasal drops, or aerosols. Formulations for parenteral administration may, for example, contain excipients, sterile water, or saline, polyalkylene glycols such as polyethylene glycol, oils of vegetable origin, or hydrogenated napthalenes. Formulations for inhalation may contain excipients, for example, lactose, or may be aqueous solutions containing, for example, polyoxyethylene-9-lauryl ether, glycolate and deoxycholate, or may be oily solutions for administration in the form of nasal drops, or as a gel. The concentration of the compound in the formulation will vary depending upon a number of factors, including the dosage of the drug to be administered, and the route of administration.

The aptamers of the present invention may be encapsulated within or administered with a biocompatible polymer to provide controlled release of the aptamer. The biocompatible polymer can be either a biodegradable polymer or a biocompatible non-degradable polymer which releases over time the incorporated aptamer by diffusion. The aptamer can be homogeneously or heterogeneously distributed within the biocompatible polymer. A variety of biocompatible polymers are useful in the practice of the invention, the choice of the polymer depending on the rate of drug release required in a particular treatment regimen. The aptamers can be provided in a polymeric sustained release formulation in which the amount of aptamer in the composition varies from about 0.1% to about 30%, from about 0.1% to about 10%, or from about 0.5% to about 5% (w/w).

Non-limiting representative synthetic, biodegradable polymers include, for example: polyamides such as poly (amino acids) and poly (peptides); polyesters such as poly (lactic acid), poly (glycolic acid), poly (lactic-co-glycolic acid), and poly (caprolactone); poly (anhydrides); polyorthoesters; polycarbonates; and chemical derivatives thereof (substitutions, additions of chemical groups (e.g., alkyl, alkylene), hydroxylations, oxidations, and other modifications routinely made by those skilled in the art), copolymers and mixtures thereof. The degradable sustained released composition can have a half-life for the release of the anti-VEGF aptamer of greater than one week, two weeks, three weeks, one month, two months, three months, or four months when placed on the sclera of an eye.

The aptamer can also be encapsulated within a biocompatible non-degradable polymer. Non-limiting representative non-degradable polymers include polysaccharides; polyethers, such as poly (ethylene oxide), poly (ethylene glycol), and poly (tetramethylene oxide); vinyl polymers, such as polyacrylates, acrylic acids, poly (vinyl alcohol), poly (vinyl pyrolidone), and poly (vinyl acetate); polyurethanes; cellulose-based polymers, such as cellulose, alkyl cellulose, hydroxyalkyl cellulose, cellulose ethers, cellulose esters, nitrocellulose, and cellulose acetates; polysiloxanes and other silicone derivatives. Alternatively, the aptamers can be encapsulated within liposomal formulations.

Useful polymeric sustained released compositions are a solid particulate having an average diameter of less than 400 μm, 200 μm, 100 μm, or 50 μm.

In certain instances, diffusion of the compositions of the invention may be facilitated by excipients such as salts, sugars or alcohols. The compound may be optionally administered as a pharmaceutically acceptable salt, such as a non-toxic acid addition salts or metal complexes that are commonly used in the pharmaceutical industry. Non-limiting examples of acid addition salts include quaternary ammonium salts; organic acids such as acetic, lactic, pamoic, maleic, citric, malic, ascorbic, succinic, benzoic, palmitic, suberic, salicylic, tartaric, methanesulfonic, toluenesulfonic, trifluoroacetic acids or hyaluronic acid and chemically derivatized versions thereof and the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Treatment of Age-Related Macular Degeneration

For the treatment of age-related macular degeneration, an aptamer of the invention is dissolved in sterile distilled water at a concentration of 5 mg/ml to 30 mg/ml. The resulting solution is loaded into a syringe, at a volume of 100 μl. The physician inserts the syringe into the vitreous of the patient's eye and injects the solution slowly (about 10 seconds), and then withdraws the syringe. This treatment is carried out for the duration of the patient's life, at various intervals (e.g., three-month intervals).

Alternatively, the aptamer is formulated into a polymeric sustained release formulation, e.g., biodegradable microspheres. Such aptamer containing polymers can be prepared using known methods. See, for example, Carrasquillo et al., J. Pharm. Pharmacol. 53:115 (2001). Desirably, the polymeric sustained release formulations are placed on the sclera of the eye of the mammal. The sustained release formulations may also be delivered by, for example, by placement on the sclera, by intravitreal injection, by subconjuntival injection or by intravenous injection. For subconjunctival injection, the patient's conjunctiva (cul de sacs) may be sterilized with topical antibiotic and by scrubbing and draping the face and lashes and lids. Local anesthesia may be also be given via subconjunctival injection of xylocaine in conjunction with the aptamer.

The following examples illustrate some preferred modes of practicing the present invention, but are not intended to limit the scope of the claimed invention. Alternative materials and methods can be utilized to obtain similar results.

### EXAMPLE 1

### Synthesis of Anti-VEGF Aptamer

An oligonucleotide having 5′-5′ and 3′-3′ inverted nucleotide caps was synthesized at a 100 μmole scale on an Akta oligonucleotide synthesizer (Pharmacia) using the standard RNA synthesis template. The support material used was CPG (approx. 700 μ pore size) loaded with an inverted T, which was attached to the support via the 5′ hydroxyl of the thymidine. This support was purchased from Prime Synthesis.

Oligonucleotide 1, shown below, was prepared.

| | |
|---|---|
| dT5′-5′C_{f}GₘGₘAᵣAᵣU_{f}C_{f}AₘGₘU_{f}GₘAₘAₘU_{f}Gₘ | (SEQ ID NO:1) |
| C_{f}U_{f}U_{f}AₘU_{f}AₘC_{f}AₘU_{f}C_{f}C_{f}Gₘ3′-3′dT | |

### Oligonucleotide I

In Oligonucleotide 1, Gₘ represents 2′-methoxyguanylic acid; Aₘ represents 2′-methoxyadenylic acid; C_{f} represents 2′-fluorocytidylic acid; U_{f} represents 2′-fluorouridylic acid; Aᵣ, represents riboadenylic acid; and dT represents deoxyribothymidylic acid. The oligonucleotide was synthesized using between 2 and 4 equivalents of phosphoramidites (2′ fluoro U, 2′ fluoro C (acetyl), 2′methoxy A (benzoyl), 2′ methoxy G (isobutyl), and 2′ TBDMS protected ribosyl A (benzoyl) at approx. 0.15 M concentration and 0.6 M ethyl thiotetrazole in acetonitrile. After the phosphoramidite coupling step the material was oxidized, capped and detritylated using standard reagents and conditions.

The crude oligonucleotide was deprotected in concentrated ammonia at 40° C. for six hours. This solution was filtered and washed with three equal volumes of DMSO. The resulting filtrate was cooled in an ice bath and treated with an HF-TEA solution. This mixture was heated at 40° C. for 1 hour. This solution was then quenched with an equal volume of 0.5 M NaOAc and the pH adjusted to about 7.0. This material was purified by anion exchange chromatography on a strong anion exchange (Q) column at approx. 75° C. using a linear gradient of 1 M NaCl in 20 mM sodium phosphate. Product fractions were combined and desalted on a polymeric reversed phase column. Desalted product was lyophilized. The lyophilized product was analyzed by heated anion exchange (Dionex column) chromatography and by MALDI mass spectroscopy.

### EXAMPLE 2

### IC₅₀ Testing for Anti-VEGF Aptamer

The ability of anti-VEGF aptamers to bind to human vascular endothelial growth factor (VEGF) was determined using a competitive binding ELISA-like assay. In this assay, recombinant VEGF₁₆₅ is bound to the wells of a 96-well plate (Quadra 96 Plus). Following blocking of nonspecific reactive sites on the plate, a matrix of the test aptamer and a biotinylated competitor, the DNA oligonucleotide shown below, were added.

| | |
|---|---|
| 5′-XXCCCGTCTTCCAGACAAGAGTGCAGGG-3′ | (SEQ ID NO:1) |

“X” represent a biotin moiety in the above representation. Both the biotinylated competitor and the test aptamer compete for binding sites on the immobilized VEGF. Following the removal of the unbound biotinylated competitor and unbound test aptamer, the amount of biotinylated competitor remaining is detected using a chemiluminescence reaction. The entire plate was immediately read on a luminometer (Victor 2). The amount of bound biotinylated competitor is inversely related to the amount of bound test aptamer. This method was used to assay the oligonucleotide 1 (see Example 1), which has an IC₅₀ of 3.277 nM.

### EXAMPLE 3

### Stability of Anti-VEGF Aptamer

The stability of the 5′-5′- and 3′-3′-capped anti-VEGF aptamers to exonuclease digestion in a range of biological fluids is assessed, e.g., in fetal calf serum, in human serum, in human plasma, and in human synovial fluid. Convenient in vitro assays for measuring oligonucleotide stability against in vivo (physiological) nuclease degradation are known in the art and described in the literature (see, e.g., Biegelman et al. (1995) J. Biol. Chem. 270: 25702-8; Uhlmann et al. (1997) Antisense Nucleic Acid Drug Dev. 7: 345-50; and Pieken et al. (1991) Science 253: 314-7).

Briefly, the aptamer oligonucleotide to be analyzed is first labeled using methods known in the art, e.g., by 5′-end-labeling (at the 3′-3′ cap's free 5′ end) with T4 polynucleotide kinase and [γ-³²P]ATP. For internal labeling, the capped anti-VEGF aptamers are first synthesized in two halves, and the 3′-half-aptamer portion is 5′-end-labeled using T4 polynucleotide kinase and [γ-³²P]ATP, and is ligated to the 5′-half-aptamer portion using, e.g. T4 RNA ligase. The labeled capped anti-VEGF aptamers aptamers are isolated from half-aptamers and unincorporated label is removed by gel electrophoresis.

Next, the stability of the labeled, 5′-5′ and 3′-3′ capped VEGF aptamer in various biological fluids is determined. Five hundred pmol of gel-purified 5′-end-labeled or internally labeled capped aptamer is ethanol-precipitated and then resuspended in 20 μl of appropriate fluid (human serum, human plasma, human synovial fluid, or fetal calf serum) by vortexing for 20 seconds at room temperature. Samples are placed at 37° C., and 2 μl aliquots are withdrawn at regular time points from 30 seconds to 72 hrous. Aliquots are quenched by the addition of 20 μl of 95% formamide, 0.5×TBE (50 mM Tris, 50 mM borate, 1 mM EDTA) and are frozen prior to loading onto a gel. The VEGF-aptamers aliquots are then size-fractionated by electrophoresis in 20% acrylamide, 8 M urea gels. Gels are imaged on a Molecular Dynamics PhosphorImager, and the stability half-life (t) for each ribozyme is calculated from exponential fits of plots of the percentage of intact ribozyme versus the time of incubation. The results with the 5′-5′ and 3′-3′ capped VEGF aptamers are compared to those obtained with control aptamers (e.g. non-capped anti-VEGF aptamer of the same sequence or an anti-VEGF aptamer having only one capped end (i.e., a 3′-3′- or a 5′-5′-singly capped anti-VEGF aptamer) and demonstrate that the 5′-5′ and 3′-3′ doubly capped aptamers are more stable, and therefore therapeutically effective, than the corresponding noncapped or singly capped forms of the aptamer.

In addition, the stability of the anti-VEGF aptamer n a pre-administration preparation, e.g., a product sample, is measured to determine stability. Briefly, the integrity of the capped anti-VEFG aptamer is determined by a time-based stability study that parallels real-life conditions so that an informed judgment may be established on the capability of the drug product for operational use. A solution of drug product at an approximate concentration of 30 mg/ml and vehicle matrix solution are subjected to 37° C. and 90% relative humidity over 42 days. Aliquots of the solutions are removed at 2, 7, 14, 28 and 42 days and diluted and analyzed by competitive plate binding assay (described in Example 2, above) and HPLC chromatography. Chromatographic results provide a quantification of the amount of active anti-VEGF aptamer in the samples, as well as the total amount of impurities and/or degradants versus the active ingredient. Competitive plate binding results provide IC₅₀ data to establish inhibition of binding to recombinant human VEGF₁₆₅ by the active ingredient.

Equivalents.

While this invention has been particularly shown and described with references to preferred embodiments thereof, it will be understood by those skilled in the art that various changes in form and details can be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

1. An aptamer comprising 5′-5′ and 3′-3′ inverted nucleotide caps.

2. The aptamer of claim 1, wherein the aptamer is an RNA aptamer.

3. The aptamer of claim 1, wherein the aptamer is a DNA aptamer.

4. An anti-VEGF aptamer comprising 5′-5′ and 3′-3′ inverted nucleotide caps.

5. The anti-VEGF aptamer of claim 4, wherein the aptamer is an RNA aptamer.

6. The anti-VEGF aptamer of claim 4, wherein the aptamer is a DNA aptamer.

7. The anti-VEGF aptamer of claim 4, comprising the sequence GAAGAAUUGG (SEQ ID NO 2), wherein each C, A, G and U is a naturally occurring or modified nucleoside.

8. The anti-VEGF aptamer of claim 4, comprising the sequence UUGGACGC (SEQ ID NO 3), wherein each C, A, G and U is a naturally occurring or modified nucleoside.

9. The anti-VEGF aptamer of claim 4, comprising the sequence GUGAAUGC (SEQ ID NO 4), wherein each C, A, G and U is a naturally occurring or modified nucleoside.

10. The aptamer of claim 4, further described by formula I:

| | |
|---|---|
| X-5′-5′-CGGAAUCAGUGAAUGCUUAUACAUCCG- | (SEQ ID NO:1) |
| 3′-3′-X | |

I
wherein C, G, A, and U represent their respective cytidylic, guanylic, adenylic, and uridylic acid nucleotides, X-5′-5′ is an inverted nucleotide capping the 5′ terminus of the aptamer and 3′-3′-X is an inverted nucleotide capping the 3′ terminus of the aptamer, and the remaining nucleotides are sequentially linked via 5′-3′ phosphodiester linkages.

11. The aptamer of claim 10, wherein each of said nucleotides, individually, comprise a 2′ ribosyl substituent selected from the group consisting of OH, H, O(C₁₋₁₀ alkyl), O(C₁₋₁₀ alkenyl), F, N₃, and NH₂.

12. The aptamer of claim 11, further described by formula II:

| | |
|---|---|
| T_{d}-5′-5′-C_{f}GₘGₘAᵣAᵣU_{f}C_{f}AₘGₘU_{f}GₘAₘAₘU_{f} | (SEQ ID NO 1) |
| GₘC_{f}U_{f}U_{f}AₘU_{f}AₘC_{f}AₘU_{f}C_{f}C_{f}Gₘ 3′-3′-T_{d} | |

II
wherein Gₘ represents 2′-methoxyguanylic acid, Aₘ represents 2′-methoxyadenylic acid, C_{f} represents 2′-fluorocytidylic acid, U_{f} represents 2′-fluorouridylic acid, Aᵣ represents riboadenylic acid, and T_{d} represents deoxyribothymidylic acid.

13. A pharmaceutical composition comprising an effective amount of an aptamer comprising 5′-5′ and 3′-3′ inverted nucleotide caps, together with a pharmaceutically acceptable carrier or diluent.

14. The pharmaceutical composition of claim 13, wherein the aptamer is an RNA aptamer.

15. The pharmaceutical composition of claim 13, wherein the aptamer is a DNA aptamer.

16. A pharmaceutical composition comprising an effective amount of an anti-VEFG aptamer having 5′-5′ and 3′-3′ inverted nucleotide caps, together with a pharmaceutically acceptable carrier or diluent.

17. The pharmaceutical composition of claim 16, wherein the anti-VEGF aptamer is an RNA aptamer.

18. The pharmaceutical composition of claim 16, wherein the anti-VEGF aptamer is a DNA aptamer.

19. The pharmaceutical composition of claim 16, wherein the anti-VEGF aptamer comprises the sequence GAAGAAUUGG (SEQ ID NO 2), and each C, A, G and U is a naturally occurring or modified nucleoside.

20. The pharmaceutical composition of claim 16, wherein the anti-VEGF aptamer comprises the sequence UUGGACGC (SEQ ID NO 3), and each C, A, G and U is a naturally occurring or modified nucleoside.

21. The pharmaceutical composition of claim 16, wherein the anti-VEGF aptamer comprises the sequence GUGAAUGC (SEQ ID NO 4), and each C, A, G and U is a naturally occurring or modified nucleoside.

22. The pharmaceutical composition of claim 16, wherein the anti-VEGF aptamer is further described by formula I:

| | |
|---|---|
| X-5′-5′-CGGAAUCAGUGAAUGCUUAUACAUCCG- | (SEQ ID NO:1) |
| 3′-3′-X | |

I
wherein C, G, A, and U represent their respective cytidylic, guanylic, adenylic, and uridylic acid nucleotides, X-5′-5′ is an inverted nucleotide capping the 5′ terminus of the aptamer and 3′-3′-X is an inverted nucleotide capping the 3′ terminus of the aptamer, and the remaining nucleotides are sequentially linked via 5′-3′ phosphodiester linkages.

23. The pharmaceutical composition of claim 22, wherein each of said nucleotides, individually, comprise a 2′ ribosyl substituent selected from the group consisting of OH, H, O(C₁₋₁₀ alkyl), O(C₁₋₁₀ alkenyl), F, N₃, and NH₂.

24. The pharmaceutical composition of claim 23, wherein the anti-VEGF aptamer is further described by formula II:

| | |
|---|---|
| T_{d}-5′-5′-C_{f}GₘGₘAᵣAᵣU_{f}C_{f}AₘGₘU_{f}GₘAₘAₘU_{f} | (SEQ ID NO:1) |
| GₘC_{f}U_{f}U_{f}AₘU_{f}AₘC_{f}AₘU_{f}C_{f}C_{f}Gₘ 3′-3′-T_{d} | |

II
wherein Gₘ represents 2′-methoxyguanylic acid, Aₘ represents 2′-methoxyadenylic acid, C_{f} represents 2′-fluorocytidylic acid, U_{f} represents 2′-fluorouridylic acid, Aᵣ represents riboadenylic acid, and T_{d} represents deoxyribothymidylic acid.

25. A composition for the sustained release of an aptamer comprising an effective amount of an aptamer having 5′-5′ and 3′-3′ inverted nucleotide caps and a biocompatible polymer which allows for the release of the aptamer.

26. The composition of claim 25, wherein the aptamer is an RNA aptamer.

27. The composition of claim 25, wherein the aptamer is a DNA aptamer.

28. A composition for the sustained release of an aptamer comprising an effective amount of an anti-VEGF aptamer having 5′-5′ and 3′-3′ inverted nucleotide caps and a biocompatible polymer which allows for the release of the aptamer.

29. The composition of claim 28, wherein the anti-VEGF aptamer is an RNA aptamer.

30. The composition of claim 28, wherein the anti-VEGF aptamer is a DNA aptamer.

31. The composition of claim 28, wherein the anti-VEGF aptamer comprises the sequence GAAGAAUUGG (SEQ ID NO 2), and each C, A, G and U is a naturally occurring or modified nucleoside.

32. The composition of claim 28, wherein the anti-VEGF aptamer comprises the sequence UUGGACGC (SEQ ID NO 3), and each C, A, G and U is a naturally occurring or modified nucleoside.

33. The composition of claim 28, wherein the anti-VEGF aptamer comprises the sequence GUGAAUGC (SEQ ID NO 4), and each C, A, G and U is a naturally occurring or modified nucleoside.

34. The composition of claim 28, wherein the anti-VEGF aptamer is further described by formula I:

| | |
|---|---|
| X-5′-5′-CGGAAUCAGUGAAUGCUUAUACAUCCG- | (SEQ ID NO:1) |
| 3′-3′-X | |

I
wherein C, G, A, and U represent their respective cytidylic, guanylic, adenylic, and uridylic acid nucleotides, X-5′-5′ is an inverted nucleotide capping the 5′ terminus of the aptamer and 3′-3′-X is an inverted nucleotide capping the 3′ terminus of the aptamer, and the remaining nucleotides are sequentially linked via 5′-3′ phosphodiester linkages.

35. The composition of claim 34, wherein each of said nucleotides, individually, comprise a 2′ ribosyl substituent selected from the group consisting of OH, H, O(C₁₋₁₀ alkyl), O(C₁₋₁₀ alkenyl), F, N₃, and NH₂.

36. The composition of claim 35, wherein the anti-VEGF aptamer is further described by formula II:

| | |
|---|---|
| T_{d}-5′-5′-C_{f}GₘGₘAᵣAᵣU_{f}C_{f}AₘGₘU_{f}GₘAₘAₘU_{f} | (SEQ ID NO:1) |
| GₘC_{f}U_{f}U_{f}AₘU_{f}AₘC_{f}AₘU_{f}C_{f}C_{f}Gₘ3′-3′-T_{d} | |

II
wherein Gₘ represents 2′-methoxyguanylic acid, Aₘ represents 2′-methoxyadenylic acid, C_{f} represents 2′-fluorocytidylic acid, U_{f} represents 2′-fluorouridylic acid, Aᵣ represents riboadenylic acid, and T_{d} represents deoxyribothymidylic acid.

37. The composition of claim 28, wherein said aptamer comprises from 0.1% (w/w) to 30% (w/w) of said composition.

38. The composition of claim 37, wherein said aptamer comprises from 0.1% (w/w) to 10% (w/w) of said composition.

39. The composition of claim 38, wherein said aptamer comprises from 0.5% (w/w) to 5% (w/w) of said composition.

40. The composition of claim 28, further comprising a stabilizing agent selected from the group consisting of saccharides, poly alcohols, proteins and hydrophilic polymers.

41. The composition of claim 28, wherein said biocompatible polymer is degradable under physiological conditions.

42. The composition of claim 41, wherein said degradable polymer is selected from the group consisting of polycarbonates, polyanhydrides, polyamides, polyesters, polyorthoesters, bioerodable hydrogels, and copolymers and mixtures thereof.

43. The composition of claim 42, wherein said polyester is selected from the group consisting of poly(lactic acid), poly(glycolic acid), poly(lactic acid-co-glycolic acid), polycaprolactone, blends thereof, and copolymers thereof.

44. The composition of claim 42, wherein said polymer comprises poly(lactic acid-co-glycolic acid).

45. The composition of claim 28, wherein said biocompatible polymer is a non-degradable polymer.

46. The composition of claim 45, wherein the non-degradable polymer is a silicone derivative.

47. The composition of claim 45, wherein the non-degradable polymer is selected from the group consisting of polysaccharides, polyether, vinyl polymer, polyurethane, cellulose-based polymer, and polysiloxane.

48. The composition of claim 47, wherein the polyether is selected from the group consisting of poly (ethylene oxide), poly (ethylene glycol), and poly (tetramethylene oxide).

49. The composition of claim 47, wherein the vinyl polymer is selected from the group consisting of polyacrylates, acrylic acids, poly (vinyl alcohol), poly (vinyl pyrolidone), and poly (vinyl acetate).

50. The composition of claim 47, wherein the cellulose-based polymer is selected from the group consisting of cellulose, alkyl cellulose, hydroxyalkyl cellulose, cellulose ethers, cellulose esters, nitrocellulose, and cellulose acetates.

51. The composition of claim 28, wherein said composition is a solid particulate having an average diameter of less than 400 μm.

52. The composition of claim 51, wherein said composition is a solid particulate having an average diameter of less than 200 μm.

53. The composition of claim 52, wherein said composition is a solid particulate having an average diameter of less than 100 μm.

54. A composition for the sustained release of an aptamer comprising an effective amount of an anti-VEGF aptamer having 5′-5′ and 3′-3′ inverted caps and a biocompatible polymer which is degradable under physiological conditions.

55. The composition of claim 54, wherein the half-life for the release of said aptamer on the sclera of an eye is greater than 1 month.

56. The composition of claim 55, wherein the half-life for the release of said aptamer on the sclera of an eye is greater than 2 months.

57. The composition of claim 56, wherein the half-life for the release of said aptamer on the sclera of an eye is greater than 4 months.


SEQUENCE LISTING (11 sequences)

SEQ ID NO: 1 - RNA, 27 nt, Artificial Sequence
  Description of Artificial Sequence Synthetic anti-VEGF aptamer
cggaaucagugaaugcuuauacauccg

SEQ ID NO: 2 - RNA, 10 nt, Artificial Sequence
  Description of Artificial Sequence Synthetic anti-VEGF aptamer
gaagaauugg

SEQ ID NO: 3 - RNA, 8 nt, Artificial Sequence
  Description of Artificial Sequence Synthetic anti-VEGF aptamer
uuggacgc

SEQ ID NO: 4 - RNA, 8 nt, Artificial Sequence
  Description of Artificial Sequence Synthetic anti-VEGF aptamer
gugaaugc

SEQ ID NO: 5 - RNA, 29 nt, Artificial Sequence
  Description of Artificial Sequence Synthetic anti-VEGF aptamer
uaggaagaauuggaagcgcauuuuccucg

SEQ ID NO: 6 - RNA, 29 nt, Artificial Sequence
  Description of Artificial Sequence Synthetic anti-VEGF aptamer
aacggaagaauuggauacguagcaugcgu

SEQ ID NO: 7 - RNA, 29 nt, Artificial Sequence
  Description of Artificial Sequence Synthetic anti-VEGF aptamer
gaaccgauggaauuuuuggacgcucgccu

SEQ ID NO: 8 - RNA, 30 nt, Artificial Sequence
  Description of Artificial Sequence Synthetic anti-VEGF aptamer
uaaccgaauugaaguuauuggacgcuaccu

SEQ ID NO: 9 - RNA, 30 nt, Artificial Sequence
  Description of Artificial Sequence Synthetic anti-VEGF aptamer
agaaucagugaaugcuuauaaaucucgcgu

SEQ ID NO: 10 - RNA, 30 nt, Artificial Sequence
  Description of Artificial Sequence Synthetic anti-VEGF aptamer
aaucagugaaugcuuauacauccgcucggu

SEQ ID NO: 11 - DNA, 26 bp, Artificial Sequence
  Description of Artificial Sequence Synthetic oligonucleotide
cccgtcttccagacaagagtgcaggg